(19) United States
(12) Patent Application Publication
(10) Pub. No.: US 2016/0184588 A1
(43) Pub. Date: Jun. 30, 2016

(54) Combined Esophageal Temperature Monitor and Pacing Device

(71) Applicant: Catheter Robotics, Inc., Budd Lake, NJ (US)

(72) Inventors: Robert Locke, San Clemente, CA (US); David Jenkins, Flanders, NJ (US)

(21) Appl. No.: 14/982,249

(22) Filed: Dec. 29, 2015

Related U.S. Application Data
(60) Provisional application No. 62/097,865, filed on Dec. 30, 2014.

(51) Int. Cl. A61N 1/365; A61N 1/37; A61N 1/05; A61B 5/0452; A61B 5/042

(52) U.S. Cl. CPC A61N 1/3655; A61B 5/0452; A61B 5/0422; A61N 1/0517; A61N 1/3702

(57) ABSTRACT

The methods, devices, and systems of the various embodiments may include an esophageal probe including and elongate tube, at least one pair of pacing electrodes and at least one temperature sensor. The elongate tube may include a proximal end for coupling to a handle and a distal end for insertion into an esophagus of a patient. The pair of pacing electrodes may be disposed at the distal end of the elongate tube, along with the temperature sensor. The temperature monitoring equipment may monitor the temperatures sensor, enable pacing to be conducted using the pair of pacing electrodes when measured temperature is within one or more thresholds, and preventing pacing from being conducted when measured temperature is not within the one or more thresholds.

### RELATED APPLICATIONS

This application claims the benefit of priority to U.S. Provisional Patent Application No. 62/097,865 entitled “Combined Esophageal Temperature Monitor and Pacing Device” filed Dec. 30, 2014, the entire contents of which are hereby incorporated by reference.

### BACKGROUND

Esophageal temperature monitors have been around for some time. An example of one such device is that manufactured by CIRCA Scientific, Inc. of Englewood, Colo. It is a probe inserted either orally or nasally and is intended to have one or more sensors in contact with the wall of the esophagus to measure temperature. One procedure in which temperature is measured is a therapeutic procedure involving ablation of the inner tissue of the heart, where the tissue is heated up to ablate, or deaden, the tissue so it no longer conducts arrhythmic electrical signals. Such ablation could heat up tissue quite close to the esophagus, with the potential of unwanted damage to the esophagus. Thus, temperature monitoring is utilized as a safety measure to cease the cardiac ablation if the esophageal temperature rises above a certain point.

Esophageal electrical pacing has also been around for some time. It is used as a non invasive means for applying electrical stimulation to the heart. Much lower output can be used in the esophageal pacing than externally through the chest. The esophageal pacing device is essentially a catheter inserted through the mouth or nose.

### SUMMARY

The methods, devices, and systems of the various embodiments may include an esophageal probe including and elongate tube, a pair of pacing electrodes and a temperature sensor. The elongate tube may include a proximal end for coupling to a handle and a distal end for insertion into an esophagus of a patient. The pair of pacing electrodes may be disposed at the distal end of the elongate tube, along with the temperature sensor.

In various embodiments the temperature sensor may be disposed proximal to the pair of pacing electrodes. The pair of pacing electrodes may be configured of a material selected from the group including platinum, gold, stainless steel, other conductive material and alloys of any combination thereof. The pair of pacing electrodes may include two or more pairs of pacing electrodes. The temperature sensor may include two or more temperature sensors. The esophageal probe may include a circuit coupled to the temperature sensor and the pair of pacing electrodes and configured to isolate the pair of pacing electrodes in response to a temperature measured by the temperature sensor exceeding a high-temperature threshold. A circuit coupled to the temperature sensor and the pair of pacing electrodes may be configured to isolate the pair of pacing electrodes in response to a temperature measured by the temperature sensor falling below a low-temperature threshold. In addition, the esophageal probe may be manufactured as a single use disposable.

Various embodiments include a method of treating a patient for an arrhythmia. The method may include inserting an esophageal probe into an esophagus of the patient. A distal end of the esophageal probe may be disposed adjacent the patient's heart, with a pair of electrodes and a temperature sensor disposed on the distal end of the esophageal probe. A temperature of the patient may be determined using the temperature sensor. The patient's heart may be paced by applying an electrical signal to the pair of electrodes in response to the temperature meeting a predetermined threshold. Optionally, the activity of the patient's heart may be monitored using the pair of electrodes. In addition, conduction timing or a conduction sequence of the patient's heart may be mapped. Thus, the patient may be diagnosed based on the conduction timing or the conduction sequence.

Various embodiments include a system including an esophageal probe, a temperature monitoring equipment, a radio frequency (RF) generator, and a circuit. The esophageal probe may include a pair of pacing electrodes and a temperature sensor. The temperature monitoring equipment may be configured to electrically couple to the temperature sensor of the esophageal probe and determine a temperature based on signals received from the temperature sensor. The RF generator may be configured to be electrically coupled to the pair of pacing electrodes and provide RF signals to the pair of pacing electrodes. The circuit may be coupled to an output of the temperature monitoring equipment and configured to interrupt RF signals from the RF generator to at least one of the pair of pacing electrodes when the determined temperature exceeds a predetermined threshold. The predetermined threshold may be a high-tempera threshold or a low-temperature threshold.

### BRIEF DESCRIPTION OF THE DRAWINGS

The accompanying drawings, which are incorporated herein and constitute part of this specification, illustrate exemplary embodiments of the invention, and together with the general description given above and the detailed description given below, serve to explain the features of the invention.

FIG. 1 is a component block diagram of a combined esophageal temperature monitor and pacing device according to various embodiments.

FIG. 2 is a component block diagram of an esophageal probe according to various embodiments.

FIG. 3 is a process flow diagram of a method of treatment using a combined esophageal temperature monitor and pacing device according to various embodiments.

### DETAILED DESCRIPTION

The various embodiments include an esophageal probe that incorporates pacing or heart stimulating electrodes in addition to sensors (e.g., temperature sensing). The esophageal probe according to various embodiments provides an esophageal monitor that not only monitors the temperature of a patient, but also allows the health practitioner to pace or stimulate the heart if needed through the same device. The various embodiments eliminate one invasive catheter that might otherwise be used during an electrophysiology procedure.

In particular, the pacing electrodes may be placed on the probe and configured such that they have the greatest potential to be in contact with the wall of the esophagus, similar to how sensors of the probe are positioned. The pacing electrodes may either be as bi-polar or mono-polar electrodes, and in an embodiment an esophageal probe may include some of both polar and mono-polar electrodes. In the various embodiments, the pacing electrodes may be individually coupled to a radio frequency (RF) generator with selection circuitry to enable individual or multiple electrodes to be selected for use, thereby allowing the health practitioner to pace through any one of several electrodes on the probe.

In an embodiment, in addition to being configured for pacing, the electrodes may be able configured and coupled to electrophysiology monitoring equipment to sense heart electrical activity. This embodiment gives the health provider a new advantage in that he or she may receive additional information regarding the heart beat and the timing of electrical activation in heart tissue close to the esophagus. This capability may allow for better diagnosis, and hence, better therapeutic application during an ablation process.

In a further embodiment, the esophageal probe may include or be configured to electrically couple to an interface circuit that is configured to shut off the RF generator if the measured patient temperature does not meet a predetermined threshold. For example, if the patient's temperature exceeds a high-temperature threshold or falls below a low-temperature threshold (which may be useful when the procedure includes cryogenic treatments) the RF generator may be shut off. The value of the predetermined threshold may be selected in order to avoid damaging and/or avoid perturbing tissue surrounding the esophageal probe 102. The predetermined threshold may be a value set by a medical practitioner, set automatically by a processor, or calculated by the processor after receiving input related to the patient. The value used as the predetermined threshold may be influenced by patient-specific parameters (e.g., age, sex, weight, height, etc.) or biometric measurements from the patient (e.g., blood pressure, heart rate, oxygen levels, etc).

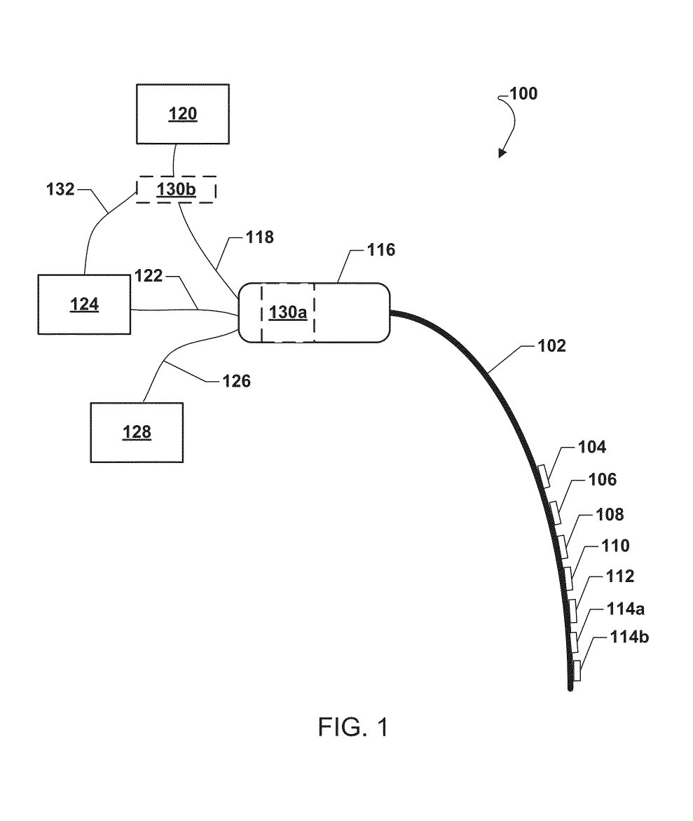

Referring to FIG. 1, a combined esophageal temperature monitor and pacing device 100 according to various embodiments may include an esophageal probe 102 formed as an elongate tube on which are positioned one or more temperature sensors 104, 108, 112 and one or more pairs of pacing electrodes 106, 110, 114a, 114b. The esophageal probe 102 may be formed as a soft, flexible shaft configured to bend to accommodate the shape of a patient's esophagus. In addition, the esophageal probe 102 may be radiopaque for visualization on fluoroscopy. The esophageal probe 102 may be sized to have a cross-sectional diameter small enough to enable both oral and nasal insertion into the patient's esophagus.

The temperature sensors 104, 108, 112 and pacing electrodes 106, 110, 114a, 114b may be arranged in various configurations along a longitudinal extent of the esophageal probe 102. Although the temperature sensors 104, 108, 112 and pacing electrodes 106, 110, 114a, 114b are illustrated as all being attached on one side of the esophageal probe 102, one or more of the temperature sensors 104, 108, 112 and pacing electrodes 106, 110, 114a, 114b may face in different directions. In addition or alternatively, the temperature sensors 104, 108, 112 and pacing electrodes 106, 110, 114a, 114b may each be formed as a band that wraps around a diameter of the elongate tube forming the esophageal probe 102 in order to provide multidirectional capabilities.

The pacing electrodes 106, 110, 114a, 114b may work in pairs to conduct electricity through tissues. A distal-most pair of pacing electrodes 114a, 114b may be disposed at a distal end of the esophageal probe 102. A distal-most temperature sensor 112 may be disposed proximal to the distal-most pair of pacing electrodes 114a, 114b, also at the end of the esophageal probe 102. The pacing electrodes 106, 110, 114a, 114b may be made from platinum, gold, stainless steel, other conductive material and alloys, or any combination thereof. The pacing electrodes 106, 110, 114a, 114b may be coated for biocompatibility and to resist corrosion. Although four pacing electrodes 106, 110, 114a, 114b are illustrated, fewer or more electrodes may be included. Distributing sufficient pacing electrodes (e.g., 106, 110, 114a, 114b) along the longitudinal extent of the esophageal probe 102 may increase the likelihood that at least one pair of pacing electrodes is properly positioned in an optimal location within the patient for performing pacing. The pacing electrodes 106, 110, 114a, 114b may be coupled via electrical connectors passing through the handle 116 to an input cable 118 that is configured to connect with a radio frequency (RF) generator 120. The RF generator 120 may be any type of pacing signal generator that includes a connector or socket for connecting to the input cable 118.

The temperature sensors 104, 108, 112 may be thermistors, thermocouples, or other temperature sensing circuits. The temperature sensors 104, 108, 112 may be electrically insulated and/or coated for biocompatibility and to resist corrosion. Although three temperature sensors 104, 108, 112 are illustrated, fewer or more temperature sensors may be included. Distributing sufficient temperature sensors (e.g., 104, 108, 112) along the longitudinal extent of the esophageal probe 102 may increase the likelihood that at least one of the temperature sensors is properly positioned in an optimal location close to a source of a change in esophageal temperature, such as adjacent to an active pacing electrode. The temperature sensors 104, 108, 112 may be connected via wires passing through a handle 116 to an output cable 122 that is configured to connect with temperature monitoring equipment 124, such as temperature monitoring the equipment used with conventional esophageal temperature probes. Thus, the temperature sensors 104, 108, 112 may feed data to the temperature monitoring equipment 124, which may in-turn display, record, and/or process esophageal temperature changes.

In an embodiment, a combined esophageal temperature monitoring and pacing device 100 may also include an output cable 126 that is coupled to the pacing electrodes 106, 110, 114a, 114b and connects to electrophysiology monitoring equipment 128 configured to monitor heart activity based on signals received via the pacing electrodes 106, 110, 114a, 114b.

In an embodiment, the combined esophageal temperature monitoring and pacing device 100 may be configured and manufactured as a single use disposable device.

In an embodiment, the combined esophageal temperature monitor and pacing device 100 may also include an onboard circuit 130a that is configured to cut off power from the RF generator 120 to the pacing electrodes 106, 110, 114a, 114b when a temperature measured by one or more of the temperature sensors 104, 108, 112 exceeds a predefined threshold. Such the onboard circuit 130a may be configured to receive signals from the temperature sensors 104, 108, 112, such as by tapping into conductors coupled to the temperature sensor output (e.g., 122), and compare those signals to the predefined threshold. The onboard circuit 130a may be further configured to isolate the pacing electrodes 106, 110, 114a, 114b from the RF generator 120 when the temperature sensor signals exceed the predefined threshold. The predefined threshold may be a value corresponding to a high patient temperature beyond which it is not safe to continue a pacing therapy, and/or a high patient temperature beyond which it is not safe to continue a pacing therapy (e.g., when the procedure includes cryogenic treatments). This embodiment provides a safety feature to prevent pacing of the patient when the patient's temperature goes outside predefined thresholds where such therapy is safe

In another embodiment, a similar safety feature may be provided via an external circuit 130b that is external to, or separate from the combined esophageal temperature monitor and pacing device 100, such as an isolation box connected to the input cable 118 between the RF generator 120 and the combined esophageal temperature monitor and pacing device 100. The external circuit 130b may receive a measured temperature from the temperature monitoring equipment 124, such as via a data cable 132 or a wireless connection (not shown), and compare the measured temperature to one or more predefined thresholds. In response to the measured temperature exceeding a predefined threshold, the external circuit 130b may open a circuit or otherwise isolate the combined esophageal temperature monitor and pacing device 100 from the RF generator 120. Again, a predefined threshold may be a value corresponding to a high patient temperature beyond which it is not safe to continue a pacing therapy, and/or a high patient temperature beyond which it is not safe to continue a pacing therapy (e.g., when the procedure includes cryogenic treatments).

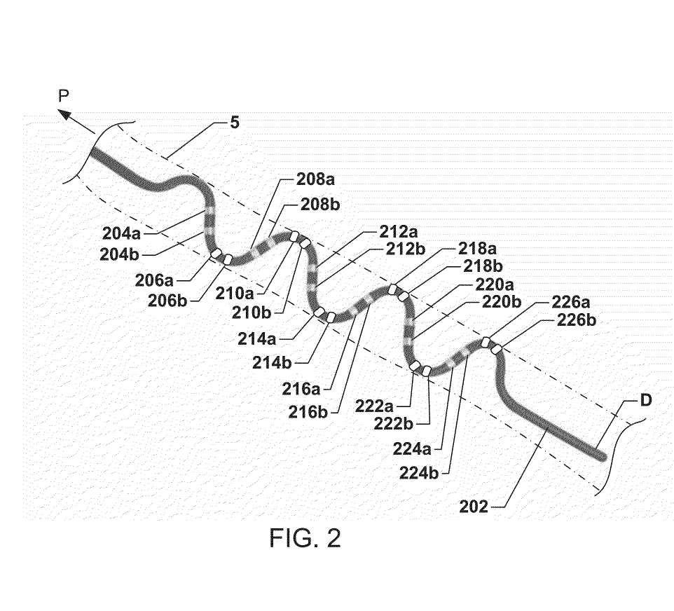

FIG. 2 illustrates a portion of an esophageal probe 202 disposed in an esophagus 5 (drawn in phantom lines) of a patient according to various embodiments. The esophageal probe 202 may include various elements described herein with reference to the esophageal probe 102. For example, the esophageal probe 202 may include an elongate tube including a proximal end P for coupling to a handle (e.g., 116 in FIG. 1) and a distal end D for insertion into the esophagus 5 of a patient. In addition, the esophageal probe 202 may include one or more pairs of pacing electrodes 206a, 206b, 210a, 210b, 214a, 214b, 218a, 218b, 222a, 222b, 226a, 226b. The esophageal probe 202 may also include one or more temperature sensors 204a, 204b, 208a, 208b, 212a, 212b, 216a, 216b, 220a, 220b, 224a, 224b.

The esophageal probe 202 may be formed as a flexible and soft shaft resiliently biased to maintain one or more S-shaped curvature. This bias enables the esophageal probe 202 to self-expand and conform to an inner shape of a patient's esophagus (e.g., 5). The esophageal probe 202 includes numerous S-shaped curves, which may help to maintain at least one pair of the pacing electrodes 206a, 206b, 210a, 210b, 214a, 214b, 218a, 218b, 222a, 222b, 226a, 226b in direct engagement with the inner walls of the esophagus 5. Similarly, positioning the temperature sensor 204a, 204b, 208a, 208b, 212a, 212b, 216a, 216b, 220a, 220b, 224a, 224b on the middle segments between the curves may help maintain the temperature sensor 204a, 204b, 208a, 208b, 212a, 212b, 216a, 216b, 220a, 220b, 224a, 224b away from the inner walls of the esophagus 5.

The esophageal probe 202 illustrated in FIG. 2 includes six pairs of pacing electrodes 206a/206b, 210a/210b, 214a/214b, 218a/218b, 222a/222b, 226a/226b. The pacing electrodes 206a, 206b, 210a, 210b, 214a, 214b, 218a, 218b, 222a, 222b, 226a, 226b may include various elements described herein with reference to the pacing electrodes 106, 110, 114a, 114b. A distal-most pair of pacing electrodes 226a/226b may be disposed at a distal end D of the esophageal probe 202. A distal-most pair of temperature sensors 224a/224b may be disposed proximal to the distal-most pair of pacing electrodes 226a/226b, also at the distal end D of the esophageal probe 102. Although six pairs of pacing electrodes 206a/206b, 210a/210b, 214a/214b, 218a/218b, 222a/222b, 226a/226b are illustrated, a fewer or greater number of electrodes may be included. Distributing sufficient pairs of pacing electrodes along the longitudinal extent of the esophageal probe 202 may increase the likelihood that at least one pair of pacing electrodes is properly positioned in an optimal location.

The esophageal probe 202 may also include six pairs of temperature sensors 204a/204b, 208a/208b, 212a/212b, 216a/216b, 220a/220b, 224a/224b. The temperature sensors 204a, 204b, 208a, 208b, 212a, 212b, 216a, 216b, 220a, 220b, 224a, 224b may include various elements described herein with reference to the temperature sensors 104, 108, 112. Although six pairs of temperature sensors 204a/204b, 208a/208b, 212a/212b, 216a/216b, 220a/220b, 224a/224b are illustrated, fewer or more temperature sensors may be included. Distributing sufficient pairs of pacing temperature sensors along the longitudinal extent of the esophageal probe 202 may increase the likelihood that at least one pair of temperature sensors is properly positioned in an optimal location within a patient.

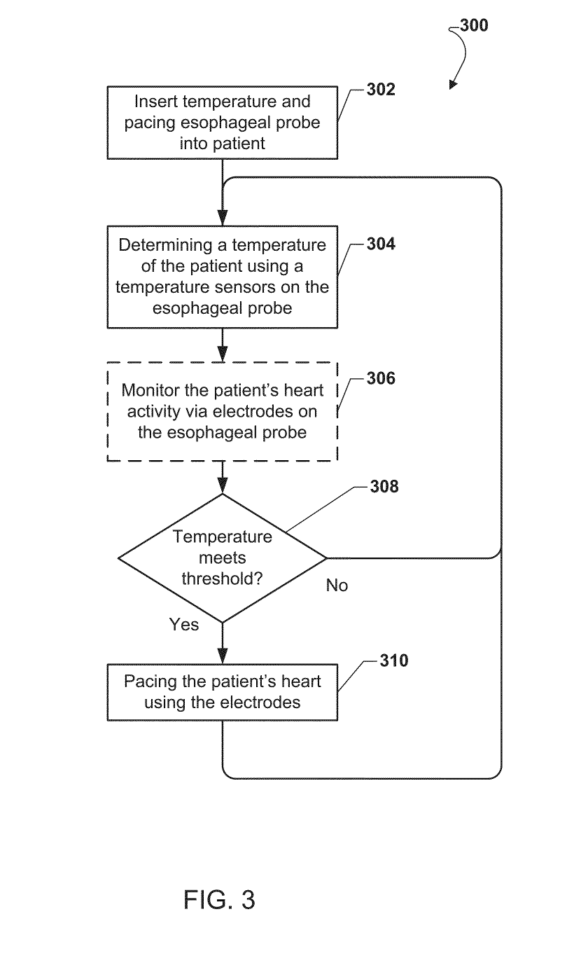

An embodiment method 300 for using an embodiment combined esophageal temperature monitor and pacing device is illustrated in FIG. 3. In block 302, a combined esophageal temperature monitoring and pacing device may be inserted into the esophagus of the patient as part of a treatment procedure. In block 304, the temperature of the patient may be monitored using temperature monitoring equipment receiving signals from the temperature sensors on the combined esophageal temperature monitor and pacing device. Optionally, activity of the patient's heart may be monitored using electrophysiology equipment receiving signals from the pacing electrodes on the combined esophageal temperature monitor and pacing device in optional block 306.

In determination block 308, the temperature monitoring equipment may determine whether the temperature of the patient meets a predetermined threshold. For example, the predetermined threshold may be a high-temperature threshold that the patient's tissues should stay below in order to avoid damage. Alternatively, the predetermined threshold may be a low-temperature threshold that the patient should stay above in order to indicate tissues healthy enough for pacing. In response to determining that the temperature of the patient exceeds the threshold (i.e., determination block 308=“Yes”), the temperature monitoring equipment may again determine the patient's temperature in block 304. In response to determining that the temperature of the patient does not exceed the threshold (i.e., determination block 308=“No”), the electrodes may be used to pace the patient's heart in block 310.

In block 310, the patient's heart may be paced by applying electrical signals via electrodes on the combined esophageal temperature monitor and pacing device. Such operations may be performed continuously, such as in a loop, throughout a medical procedure.

In summary, the various embodiments include an esophageal temperature probe, comprising an electrical pacing electrode and a temperature sensor. The pacing electrode is configured of a material selected from the group including platinum, gold, stainless steel, other conductive material and alloys of any combination thereof. The esophageal temperature probe may include two or more pacing electrodes and/or two or more temperature sensors. In an embodiment, the esophageal temperature probe may further include a circuit coupled to the temperature sensor and the pacing electrode and configured to isolate the pacing electrode in response to a temperature measured by the temperature sensor exceeding a threshold, which may be a high-temperature threshold and/or a low-temperature threshold. In an embodiment, the esophageal temperature probe may be manufactured as a single use disposable. In an embodiment, a method of treating a patient for an arrhythmia may include diagnosing the patient using multiple electrodes in the patient's esophagus using a combined esophageal temperature probe and pacing device configured for mapping conduction timing or conduction sequences of the patient's heart. In another embodiment, a medical treatment system may include an esophageal temperature probe having an electrical pacing electrode and a temperature sensor, temperature monitoring equipment configured to electrically couple to the temperature sensor of the esophageal temperature probe and determine a temperature based on signals received from the temperature sensor, and RF generator configured to electrically coupled to the pacing electrode and provide RF signals to the pacing electrode, and a circuit coupled to an output of the temperature monitoring equipment and configured to interrupt RF signals from the RF generator to the pacing electrode when the determined temperature exceeds a threshold, which may be a high-temperature threshold and/or a high-temperature threshold.

The foregoing method descriptions and the process flow diagrams are provided merely as illustrative examples and are not intended to require or imply that the blocks of various embodiments must be performed in the order presented. As will be appreciated by one of skill in the art the order of blocks in the foregoing embodiments may be performed in any order. Words such as “thereafter,” “then,” “next,” etc. are not intended to limit the order of the blocks; these words are simply used to guide the reader through the description of the methods. Further, any reference to claim elements in the singular, for example, using the articles “a,” “an” or “the” is not to be construed as limiting the element to the singular.

The preceding description of the disclosed embodiments is provided to enable any person skilled in the art to make or use the present invention. Various modifications to these embodiments will be readily apparent to those skilled in the art, and the generic principles defined herein may be applied to other embodiments without departing from the spirit or scope of the invention. Thus, the present invention is not intended to be limited to the embodiments shown herein but is to be accorded the widest scope consistent with the following claims and the principles and novel features disclosed herein.

What we claim is:

1. An esophageal probe, comprising:
an elongate tube including a proximal end for coupling to a handle and a distal end for insertion into an esophagus of a patient;
a pair of pacing electrodes disposed near the distal end of the elongate tube; and
a temperature sensor disposed near the distal end of the elongate tube.

2. The esophageal probe of claim 1, wherein the temperature sensor is disposed proximal to the pair of pacing electrodes.

3. The esophageal probe of claim 1, wherein the pair of pacing electrodes are configured of a material selected from the group including platinum, gold, stainless steel, other conductive material and alloys of any combination thereof.

4. The esophageal probe of claim 1, wherein the pair of pacing electrodes comprises two or more pairs of pacing electrodes.

5. The esophageal probe of claim 1, wherein the temperature sensor comprises two or more temperature sensors.

6. The esophageal probe of claim 1, further comprising a circuit coupled to the temperature sensor and the pair of pacing electrodes and configured to isolate the pair of pacing electrodes in response to a temperature measured by the temperature sensor exceeding a high-temperature threshold.

7. The esophageal probe of claim 1, further comprising a circuit coupled to the temperature sensor and the pair of pacing electrodes and configured to isolate the pair of pacing electrodes in response to a temperature measured by the temperature sensor falls below a low-temperature threshold.

8. The esophageal probe of claim 1, wherein the esophageal probe is manufactured as a single use disposable.

9. A method of treating a patient for an arrhythmia, comprising:
inserting an esophageal probe into an esophagus of the patient, wherein a distal end of the esophageal probe is disposed adjacent the patient's heart, wherein a pair of electrodes and a temperature sensor are disposed on the distal end;
determining a temperature of the patient using the temperature sensor; and
pacing the patient's heart by applying an electrical signal to the pair of electrodes in response to the temperature meeting a predetermined threshold.

10. The method of claim 9, further comprising:
monitoring activity of the patient's heart using the pair of electrodes.

11. The method of claim 9, further comprising
mapping a conduction timing or a conduction sequence of the patient's heart; and
diagnosing the patient based on the conduction timing or the conduction sequence.

12. A system, comprising:
an esophageal probe comprising a pair of pacing electrodes and a temperature sensor;
temperature monitoring equipment configured to electrically couple to the temperature sensor of the esophageal probe and determine a temperature based on signals received from the temperature sensor;
a radio frequency (RF) generator configured to electrically coupled to the pair of pacing electrodes and provide RF signals to the pair of pacing electrodes;
a circuit coupled to an output of the temperature monitoring equipment and configured to interrupt RF signals from the RF generator to at least one of the pair of pacing electrodes when the determined temperature exceeds a predetermined threshold.

13. The system of claim 12, wherein the threshold is a high-temperature threshold.

14. The system of claim 12, wherein the threshold is a low-temperature threshold.
